(19) United States
(12) Patent Application Publication
(10) Pub. No.: US 2024/0226017 A1
(43) Pub. Date: Jul. 11, 2024

(54) ERODIBLE TABLET

(71) Applicant: Eli Lilly and Company, Indianapolis, IN (US)

(72) Inventors: Aktham ABURUB, Carmel, IN (US); Mridula DOGRA, Zionsville, IN (US); Mohamed ElSayed Hamed ELSAYED, Fishers, IN (US); Siyuan HUANG, Indianapolis, IN (US); Phenil Jayantilal PATEL, Carmel, IN (US); Huyen Thanh TRAN, Bargersville, IN (US)

(21) Appl. No.: 18/559,467

(22) PCT Filed: May 6, 2022
(86) PCT No.: PCT/US2022/027976

§ 371 Date: Nov. 7, 2023

Related U.S. Application Data
(60) Provisional application No. 63/185,615, filed on May 7, 2021.

(51) Int. Cl. A61K 9/20; A61K 8/02; A61K 9/00; A61K 38/26

(52) U.S. Cl. CPC A61K 9/2072; A61K 8/0216; A61K 9/0053; A61K 9/2013; A61K 9/2054; A61K 38/26

(57) ABSTRACT

The present invention relates to an erodible tablet comprising a therapeutic peptide which is suitable for oral administration and, in addition, to a non-granulation process for making the erodible tablet.

### BACKGROUND OF THE INVENTION

The present invention is in the field of medicine. More particularly, the present invention relates to an erodible tablet comprising a therapeutic peptide which is suitable for oral delivery and, in addition, to a non-granulation process for making the erodible tablet. More particularly, the present invention relates to an erodible tablet comprising a pharmaceutical formulation in which the therapeutic peptide is an incretin analogue with activity at the glucagon-like peptide (GLP-1) receptor and/or at the glucose-dependent insulinotropic polypeptide (GIP) receptor. Such therapeutic peptides may also have activity at glucagon (GCG) receptor. The erodible tablets of the present invention comprise a therapeutic peptide that is a single GIP, GLP-1, or GCG receptor agonist, a dual GIP/GLP-1 receptor agonist, or a tri GIP/GLP-1/GCG receptor agonist and may be useful in the treatment of at least type 2 diabetes (T2D), obesity, nonalcoholic fatty liver disease (NAFLD) and/or nonalcoholic steatohepatitis (NASH) and/or in the prevention of cognitive decline.

Pharmaceutical compositions of such agonists are useful in the treatment of patients with at least type 2 diabetes (T2D), obesity, nonalcoholic fatty liver disease (NAFLD) and/or nonalcoholic steatohepatitis (NASH). Administration of such therapeutic peptides is by oral administration. Oral administration encourages more patient compliance and allows the patient to self-administer the therapeutic peptides.

Certain concentrations of agonist peptides are required for pharmaceutical formulations to orally deliver an effective dose to the patient. It is, therefore, important that the tablet to be administered adequately maintains physical and chemical stability of the peptide. The formulation of a therapeutic peptide into an oral formulation remains challenging and unpredictable.

The challenge and unpredictability associated with formulating therapeutic peptides such that they are suitable for oral administration is due, in part, to the numerous properties a pharmaceutical formulation must possess in order to be therapeutically viable in tablet form, whilst at the same time maintaining the therapeutic peptide's functional characteristics essential for therapeutic efficacy. In addition, the pharmaceutical formulation must also be safe for administration to, and well tolerated by, patients as well as being suitable for manufacturing and storage. The formulation must, therefore, comprise agents that provide commercially acceptable shelf-life stability, in-use stability and acceptable patient experience.

The inefficient transport of biologics, including incretin peptides, across the stomach and/or the intestinal wall has been a technology challenge for the oral delivery of therapeutics. Most of the active pharmaceutical ingredient (API) is rapidly degraded or not absorbed resulting in only around 1% systemic bioavailability. Large amounts of API are, therefore, required in order to administer an effective therapeutic dose. Most of the costly API is thus wasted, and a large tablet may be difficult for the patient to swallow.

WO 2019/149880 (Novo Nordisk) describes pharmaceutical compositions comprising a peptide, such as a GLP-1 peptide and a salt of N-(-8-(2-hydroxybenzoyl)amino)caprylic acid. The pharmaceutical compositions are prepared by a granulation method.

### BRIEF SUMMARY OF THE INVENTION

It has surprisingly been found that, despite the tablets of the present invention being produced without a granulation step, that the erodible tablets of the present invention not only have acceptable physical properties, but also achieve the desired dissolution profile and result in sufficient bioavailability following oral administration.

The production of faster-eroding tablets with high solid fraction and acceptable compression properties in the absence of microcrystalline cellulose (MCC), which is used as a compression aid especially in a non-granulation process, was unexpected. The tablet compositions of the present invention formulated without the use of MCC provide options for a wider range of erosion profile and allows bioavailability modulation for GIP/GLP-1 agonists and other peptides, whilst maintaining acceptable tablet physical properties and a smaller tablet size.

The present invention seeks to provide efficient peptide (incretin) delivery via the oral route and at the same time reduce the amount of API needed for oral efficacy.

The present invention seeks to provide an oral formulation for therapeutic peptides having agonist activity at the GIP and GLP-1 receptors that are suitable for oral administration.

The present invention also seeks to provide a process of producing an erodible tablet which reduces costs associated with API generation, whilst still allowing the convenience and compliance advantages of taking an oral pill. Such process does not include a granulation step.

In addition, the present invention seeks to provide an erodible tablet which is able to achieve efficient delivery of incretins via the oral route to achieve improved drug bioavailability, beyond currently attainable levels.

The present invention also seeks to provide a controlled erodible tablet wherein in an embodiment the tablet is slower erodible tablet using MCC and, in another embodiment, a faster-eroding tablet with a reduced or absence of MCC.

According to a first aspect of the present invention there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.75 and 0.98.

Preferably, the erodible tablet according to the present invention has an average solid fraction of the tablet between 0.8 and 0.98. More preferably, the erodible tablet according to the present invention has an average solid fraction of the tablet between 0.82 and 0.96. The erodible tablet according to the present invention has an average solid fraction of 0.82, 0.89, 0.90, 0.91, 0.92, 0.93, 0.96.

Preferably, the erodible tablet according to the present invention optionally further comprises microcrystalline cellulose (MCC). The MCC in the tablet, is preferably up to 175 mg. It is more preferably up to 169 mg. More preferably it is between 84 and 169 mg. Even more preferably, it is 84, 86, 92, 97, 110, 143, 143 and 169 mg. Most preferred is between about 30 to about 90 mg.

According to a preferred embodiment there is provided an erodible tablet according to the present invention wherein the permeation enhancer in the tablet is Sodium N-[8-(2-hydroxybenzoyl)amino]caprylate, (SNAC or Salcaprozate Sodium), Sodium Caprate (C10) or 8-(N-2-hydroxy-5-chlorobenzoyl)-amino-caprylic acid (5-CNAC). Preferably, the permeation enhancer is SNAC which is between about 300 and 600 mg in the tablet. More preferably, the SNAC in the tablet is 300 mg or 600 mg.

Alternatively, the permeation enhancer is C10 which is between 300 and 500 mg. Preferably, the C10 in the tablet is 300 mg or 500 mg. A further alternative permeation enhancer is 5-CNAC which is at about 500 mg.

According to another preferred embodiment, there is provided an erodible tablet according to the present invention wherein the lubricant is magnesium stearate. Preferably, the magnesium stearate in the tablet is between 3 and 30 mg. More preferably, the magnesium stearate in the tablet is 3.10, 4, 6.17, 6.5, 7, 8 and 9.82 mg.

Another preferred embodiment of the present invention is an erodible tablet according to the present invention wherein the therapeutic peptide in the tablet is between 1 and 50 mg. More preferably, the therapeutic tablet is in the range of 1 to 36 mg. Yet more preferably, the therapeutic peptide in the tablet is 4, 10, 24 or 36 mg. More preferably, the therapeutic peptide has agonist activity at the glucose-dependent insulinotropic polypeptide (GIP) receptor, the glucagon-like peptide-1 (GLP-1) or the glucagon (GCG) receptor. Preferably, the therapeutic peptide has agonist activity at the glucose-dependent insulinotropic polypeptide (GIP) receptor and at the glucagon-like peptide-1 (GLP-1) receptor. Even more preferably, the therapeutic peptide further has glucagon (GCG) receptor activity.

According to a preferred embodiment the therapeutic peptide is Compound 1 or Compound 2. These compounds are described in WO2020/023386. Other peptides having GIP and GLP activity are described in WO2016/111971 and WO2013/164483.

A further preferred embodiment provides an erodible tablet according to the present invention wherein the therapeutic peptide and the permeation enhancer are released concurrently. Preferably, greater than 80% concurrent release of the therapeutic peptide and permeation enhancer is achieved. More preferably, greater than 80% release of the therapeutic peptide and the permeation enhancer is achieved within 60 minutes, even more preferably 80% release of the therapeutic peptide and the permeation enhancer is achieved within 45 minutes. Most preferred is 80% release of the therapeutic peptide and the permeation enhancer is achieved within 30 minutes. Preferably, 80% release is achieved over 20 to 60 minutes, more preferably 25 to 60 minutes, even more preferably 30 to 60 minutes or 45 to 60 minutes. A preferred embodiment is where the therapeutic peptide and the permeation enhancer is released over 30 to 60 minutes. The release of the permeation enhancer is measured using HPLC.

According to an even more preferred embodiment, there is provided an erodible tablet according to the present invention which does not contain any MCC wherein the therapeutic peptide and the permeation enhancer are released over 15 to 30 minutes. Alternatively, the MCC may be reduced.

An erodible tablet according to any of the present invention is preferably film-coated with 4%±1% (w/w) coating.

According to a second aspect of the present invention, there is provided a cosmetic composition comprising an erodible tablet according to the present invention wherein the tablet is film-coated with 4%±1% (w/w) coating.

According to a preferred embodiment of the present invention, there is provided an erodible tablet according to the present invention wherein the tooling size of the tablet is about 10 to about 12 mm round.

According to a third aspect of the present invention, there is provided a method of manufacturing an erodible tablet according to the present invention comprising blending the therapeutic peptide, the permeation enhancer, the lubricant and, optionally the microcrystalline cellulose and compressing the blended constituents to achieve an average solid fraction between 0.75 and 0.98.

Preferably, the method as described above has an average solid fraction of the tablet between 0.8 and 0.98. Even more preferably the average solid fraction of the tablet is between 0.82 and 0.96. More preferably, it is 0.82, 0.89, 0.90, 0.91, 0.92, 0.93, 0.96. The process of the present invention does not, however, include a granulation step or include the use of a granulate. Preferably, the process of the present invention is a direct compression process.

According to a fourth aspect of the present invention, there is provided an erodible tablet for oral administration produced by the method described above. Preferably, the erodible tablet has an average solid fraction between 0.75 and 0.98, more preferably, between 0.82 and 0.98 and even more preferably, between 0.82 and 0.96.

According to a fifth aspect of the present invention, there is provided a method of treating diabetes comprising the steps of: administering to an individual in need thereof an erodible tablet according to the present invention. Preferably, the erodible tablet is administered once daily, twice daily, alternate days, every third day, every fourth day, every fifth day, every sixth day or once weekly. A preferred embodiment is daily administration.

According to a sixth aspect of the present invention, there is provided a method of treating obesity comprising the steps of: orally administering to an individual in need thereof an erodible tablet according to the present invention. Preferably, the erodible tablet is administered once daily, twice daily, alternate days, every third day, every fourth day, every fifth day, every sixth day or once weekly. A preferred embodiment is daily administration.

According to a seventh aspect of the present invention, there is provided an erodible tablet for use in the treatment of diabetes mellitus, dyslipidemia, fatty liver disease, metabolic syndrome, non-alcoholic steatohepatitis and obesity or prevention of cognitive decline. Preferably, the tablet is for use in the treatment of type II diabetes mellitus.

According to another preferred embodiment there is provided an erodible tablet according to the present invention for use in the treatment of obesity.

According to a preferred embodiment of the present invention, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the tablet also comprises MCC between 84 and 169 mg.

According to a further preferred embodiment of the present invention, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the tablet also comprises MCC between about 30 to about 90 mg and wherein the permeation enhancer is SNAC between 300 and 600 mg.

According to another preferred embodiment of the present invention, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the tablet also comprises MCC between about 30 to about 90 mg and wherein the permeation enhancer is SNAC between 300 and 600 mg wherein the lubricant is magnesium stearate between 3 and 30 mg.

According to a preferred embodiment of the present invention, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the tablet also comprises MCC between about 30 to about 90 mg and wherein the therapeutic peptide and the permeation enhancer are released concurrently such that greater than 80% concurrent release of the therapeutic peptide and the permeation enhancer is achieved.

According to a preferred embodiment of the present invention, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the tablet also comprises MCC between about 30 to about 90 mg wherein greater than 80% release of the therapeutic peptide and the permeation enhancer is achieved within 30 minutes.

According to a preferred embodiment of the present invention, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the tablet also comprises MCC between about 30 to about 90 mg wherein the tablet core is film coated with 4%+1% (w/w).

According to a preferred embodiment of the present invention, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the tablet also comprises MCC between about 30 to about 90 mg wherein the therapeutic peptide and the permeation enhancer are released over 30 to 60 minutes wherein the therapeutic peptide has activity at each of a glucose-dependent insulinotropic polypeptide (GIP) receptor and glucagon-like peptide-1 (GLP-1).

According to a preferred embodiment of the present invention, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the tablet also comprises MCC between about 30 to about 90 mg wherein the therapeutic peptide and the permeation enhancer are released over 30 to 60 minutes wherein the therapeutic peptide has activity at each of a glucose-dependent insulinotropic polypeptide (GIP) receptor and glucagon-like peptide-1 (GLP-1) receptor and glucagon (GCG) receptor.

According to yet another preferred embodiment, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the therapeutic peptide and the permeation enhancer are released over 15 to 30 minutes.

According to yet another preferred embodiment, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the therapeutic peptide and the permeation enhancer are released over 15 to 30 minutes and wherein the tablet core is film coated with 4%+1% (w/w).

According to yet another preferred embodiment, there is provided an erodible tablet for oral administration wherein the erodible tablet comprises a therapeutic peptide or a pharmaceutically acceptable salt thereof;
a permeation enhancer; and
a lubricant wherein the average solid fraction of the tablet is between 0.82 and 0.96 wherein the therapeutic peptide and the permeation enhancer are released over 15 to 30 minutes wherein greater than 80% concurrent release of the therapeutic peptide and the permeation enhancer is achieved.

In an embodiment the permeation enhancer is selected from the group consisting of sodium decanoate (“C10”), sodium taurodeoxycholate (“NaTDC”), lauroyl carnitine (“LC”), dodecyl maltoside (“C12-maltoside”), dodecyl phosphatidylcholine (“DPC”), sodium N-[8-(2-hydroxybenzoyl)amino]caprylate (“SNAC”) and a Rhamnolipid.

The term “GLP-1 agonist” or “a therapeutic peptide having GLP-1 activity” as used herein refers to a compound, which fully or partially activates the human GLP-1 receptor. The term GLP-1 agonist as well as the specific agonists described herein are meant to encompass salt forms thereof. The term “GLP-1 agonist” may include a GLP analogue.

In some embodiments, the term “GLP-1 analogue” refers to a peptide, or a compound, which is a variant of the human Glucagon-like peptide-1 (GLP-1(7-37). Human GLP-1(7-37) has the sequence HAEGTFTSDVSSYLEGQAAKEFIAWLVKGRG.

The term “GIP agonist” or “a therapeutic peptide having GIP activity” as used herein refers to a compound, which fully or partially activates the human GIP receptor. GIP is a 42 amino acid peptide which, like GLP-1, is also known as an incretin. GIP plays a physiological role in glucose homeostasis by stimulating insulin secretion from pancreatic beta cells in the presence of glucose.

In some embodiments, the term “GIP analogue” refers to a peptide, or a compound, which is a variant of the human GIP. Human GIP has the sequence YAEGTFISDYSIAMDKIHQQDFVNWLLAQKGKKNDWKHNITQ.

The term “GCG agonist” or a therapeutic peptide having Glucagon activity” as used herein refers to a compound, which fully or partially activates the human Glucagon receptor. Glucagon raises the concentration of glucose and fatty acids in the bloodstream.

In some embodiments, the term “GCG analogue” refers to a peptide, or a compound, which is a variant of the human GCG. Human GCG has the sequence HSQGTFTSDYSKYLDSRRAQDFVQWLMNT.

Each of the therapeutic peptides of the present invention may be modified in order to extend the half-life of the peptide or to provide desired physiologic effect.

The term “permeation enhancer” as used here refers to an agent that improves oral delivery of therapeutic peptides by increasing the passive permeability either by promoting the paracellular passage of drugs, by opening tight junctions, and/or altering epithelial membrane to increase transcellular permeation.

### DETAILED DESCRIPTION OF THE INVENTION

The present invention provides a tablet for oral administration. The tablets are erodible and comprise a therapeutic peptide or pharmaceutically acceptable salt thereof, a permeation enhancer and a lubricant wherein the average solid fraction of the tablet is between 0.75 and 0.98.

The erodible tablets of the present invention are, preferably, monolithic dosage forms, intended for concurrent release of active ingredient and permeation enhancer in the stomach, are developed. These monolithic dosage forms may take the form of uncoated tablets, or tablets with a film-coating. The tablet cores of the erodible tablets do not disintegrate, and, as a result, they release the drug and permeation enhancer via erosion.

The erodible tablets of the present invention do not require a disintegrant, such as crospovidone, that is normally used in tablet formulations in order to enable disintegration. Furthermore, the tablets of the present invention are produced by a direct compression process rather than by a granulation process. Thus, processes and methods of manufacturing described herein should be understood as not including dry granulation or milling steps except where explicitly stated otherwise.

The absence of a disintegrant combined with appropriate processing conditions such as high compression pressure (for example between 60 and 300 MPa) produces a tablet which erodes slowly resulting in concurrent slow release of the drug and permeation enhancer, rather than rapid release due to tablet disintegration.

The erodible tablets of the present invention comprising therapeutic peptide drug and a permeation enhancer (such as SNAC, 5-CNAC or C-10), added in high amounts (such as 300 mg or 600 mg permeation enhancer per tablet), are designed to erode such that greater than 80% release of the therapeutic peptide and permeation enhancer is achieved within 60 minutes. Preferably, greater than 80% release of the therapeutic peptide and permeation enhancer is achieved within 45 minutes and more preferably within 30 minutes.

The present invention also relates to a direct compression process which produces an erodible tablet according to the present invention. The compression process of the present invention has the advantage of a decreased number of unit operations compared with a granulation process. In turn, this decreases the risk of peptide instability, whether chemical or physical. It was unexpectedly found that the physical properties of the tablets were surprisingly maintained using a direct compression process. It is generally understood in the art that it is necessary to use a granulation process such as that described in EP2827845B in order to obtain therapeutic peptides containing tablets which are suitable for oral administration as the therapeutic peptides have poor physical properties, which generally necessitate use of a granulation process.

An important feature of the process is the resulting porosity of the tablet. In this regard, the compression pressure is adjusted to achieve the desired porosity. A high target compression pressure is applied and the resulting average solid fraction is between 0.75 and 0.98, preferably between 0.8 and 0.98 and even more preferably between 0.82 and 0.96. Tablet solid fraction quantifies how much of the tablet is solid i.e. not porous. It is calculated by dividing the density of a tablet by the material true density. The density of a tablet can be determined by measuring its weight and volume, where volume is determined by tablet thickness and tooling design information. True density of a powder blend is determined typically by helium pycnometry where the density excluding all the voids is measured. Alternatively, the porosity may be calculated using the calculation: Porosity=(1-Solid fraction). Porosity of the tablet is preferably between 0.04 and 0.18. More preferably, it is 0.04, 0.07, 0.08, 0.09, 0.10, 0.11, 0.14 or 0.18,

Erodible tablets comprising therapeutic peptides made by a direct compression process is exemplified using Compound 1 or Compound 2. Such agonists are GIP/GLP-1 peptide agonists.

According to a preferred embodiment of the present invention, there is provided another variation of an erodible tablet having relatively faster dissolution profile. These tablets are designed to erode within 30 minutes, by reduction of or complete removal of microcrystalline cellulose (MCC). MCC is generally used as a compression aid. Removal or reduction of microcrystalline cellulose has the advantage of producing a smaller tablet. The benefit of a smaller tablet is that it is easier to swallow and, therefore, likely to encourage more patient compliance, especially those with swallowing difficulties. These tablets have faster erosion rate relative to the tablet formulation containing a higher amount of microcrystalline cellulose and yet, surprisingly, there is no notable impact on the tablet compression profile. An erodible tablet according to the present invention comprising 10 mg of the therapeutic peptide (Compound 1 or Compound 2, as shown below) and 300 mg or 600 mg of SNAC, without addition of microcrystalline cellulose, and using magnesium stearate as a lubricant, is shown to provide the faster dissolution described above.

The erodible tablets according to the present invention may additionally have a film-coating which may be applied to both slower and faster dissolution tablet cores. The film coating is an aqueous coating solution which is applied onto the tablet core. Preferably, about a 4%±1% (w/w) coating is applied to the tablet core. The resulting cosmetically coated slower-release and faster-release erodible tablets have the advantage that they are more easily handled and swallowed by the patient as a result of the smoother finish. An example of a cosmetic film coating is Opadry Blue 03K 105008, which is composed of HPMC 2910, TiO2, Triacetin and FD&C Blue 2 Aluminum lake.

The Examples below describe erodible tablets made by a direct compression process. Preferably, the erodible tablets according to the present invention comprise a therapeutic peptide which has agonist activity at the glucose-dependent insulinotropic polypeptide (GIP) receptor, the glucagon-like peptide-1 (GLP-1) or the glucagon (GCG) receptor. More preferably, the erodible tablets according to the present invention comprise a therapeutic peptide which has agonist activity at each of a glucose-dependent insulinotropic polypeptide (GIP) receptor and glucagon-like peptide-1 (GLP-1). Preferably, the therapeutic peptide is Compound 1 or Compound 2, as defined below.

The therapeutic peptide may also have agonist activity at the GLP-1, GIP, and glucagon (GCG) receptors. The direct compression process may also be applied to other therapeutic peptides including peptides that agonise at the amylin receptor and those that agonise both the amylin and calcitonin receptors.

The present invention also provides for the co-administration of the therapeutic peptide agonist according to the present invention with one or more additional therapeutic peptide agonists or non-peptide agonists.

Other peptides agonists or non-peptide agonists may be co-formulated in the erodible tablet of the present invention. The erodible tablets of the present invention may be co-formulated comprising a therapeutic peptide with one or more agents selected from the group consisting of metformin, a thiazolidinedione, a sulfonylurea, a dipeptidyl peptidase 4 inhibitor, a sodium glucose co-transporter, a SGLT-2 inhibitor, a growth differentiation factor 15 modulator (“GDF15”), a peptide tyrosine modulator (“PYY”), a modified insulin, amylin, a dual amylin calcitonin receptor agonist, and oxyntomodulin agonist (“OXM”). In an embodiment, an erodible tablet of the present invention comprises a therapeutic peptide in a fixed dose combination with one or more agents selected from the group consisting of metformin, a thiazolidinedione, a sulfonylurea, a dipeptidyl peptidase 4 inhibitor, a sodium glucose co-transporter, a SGLT-2 inhibitor, GDF15, PYY, a modified insulin, amylin, a dual amylin calcitonin receptor agonist, and OXM.

The erodible tablets may vary in the amount of lubricant required for the compression process, typically ranging from 0.5% to 5%, and more preferably about 1 to about 5%.

It may be necessary, for optimum erodible tablet formation upon scale-up, to add the appropriate amount of microcrystalline cellulose in order to achieve a robust tablet formulation that results in concurrent release that is within the range described above in respect of the slower and faster tablets. The addition of mannitol, lactose or other diluent instead of or in addition to microcrystalline cellulose may be preferred in order to enable a robust manufacturing process whilst maintaining the desired dissolution profile.

The preparation of Compounds 1 and 2 are disclosed in PCT application number PCT/US2019/042822, publication number WO2020/023386. Compound 2 may also be prepared by the process disclosed in WO2021/034815.

### Compound 1

(SEQ ID NO: 1)
(D-Tyr)-Aib-EGTFTSDYSI-αMeL-LDKK((2-[2-(2-Amino-ethoxy)-ethoxy]-acetyl)₂-
(γ-Glu)-CO-(CH₂)₁₈-CO₂H)AQ-Aib-EFIE-αMeY-LIAGGPSSGAPPPS-NH₂

The structure of SEQ ID NO:1 is depicted below using the standard single letter amino acid codes with the exception of residues D-Tyr1, Aib2, αMeL13, K17, Aib20, αMeY25, and Ser39, where the structures of these amino acid residues have been expanded:

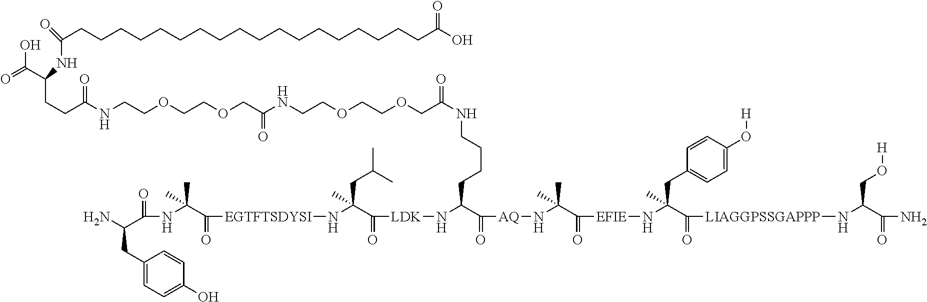

### Compound 2

(SEQ ID NO: 2)
Y-Aib-EGT-αMeF(2F)-TSD-4Pal-SI-αMeL-LD-Orn-K((2-[2-(2-Amino-ethoxy)-
ethoxy]-acetyl)₂-(γ-Glu)-CO-(CH₂)₁₆-CO₂H)AQ-Aib-EFI-(D-Glu)-αMeY-
LIEGGPSSGAPPPS-NH₂

The structure of SEQ ID NO:2 is depicted below using the standard single letter amino acid codes with the exception of residues Aib2, αMeF(2F)6, 4Pal10, αMeL13, Orn16, K17, Aib20, D-Glu24 αMeY25, and Ser39, where the structures of these amino acid residues have been expanded:

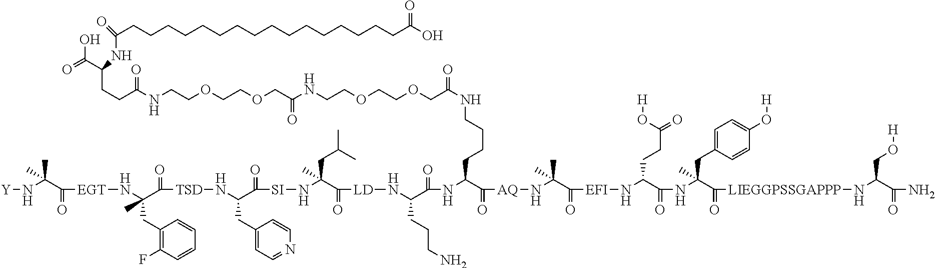

Both Compounds 1 and 2 are therapeutic peptide agonists at both the GIP and the GLP-1 receptors as described in WO2020/023386.

### Description and Examples of Solid Formulations for Oral Peptide Delivery

Monolithic dosage forms intended for concurrent release of active ingredient and permeation enhancer in stomach are developed. These monolithic dosage forms are designed as uncoated tablets, or tablets with film-coating. Tablet cores are non-disintegrating, and they release the drug and permeation enhancer via erosion.

Tablets containing peptide drug and a permeation enhancer (such as SNAC), added in high amounts (such as 300 mg or 600 mg per tablet), are designed to erode over 45 min. Whether a particular permeation enhancer is able to enhance the absorption of a peptide can not a priori be predicted. These tablets do not contain disintegrant that is normally utilized in tablet formulations and are made by direct compression. Absence of disintegrant combined with appropriate processing conditions such as high compression pressure, facilitate slow erosion of the tablets resulting in concurrent slow release of the drug and permeation enhancer, rather than rapid release due to tablet disintegration. A direct compression process is utilized in order to decrease the number of unit operations and hence decrease the risk of peptide instability whether chemical or physical. Utilizing a direct compression process is not obvious due to poor physical properties of peptides, which generally necessitate utilizing a granulation process. This is exemplified using Compound 1 and 2 as the therapeutic peptide agonists.

Another variation of such tablets, with novel composition, is developed with relatively faster dissolution profile. These tablets are designed to erode over 30 minutes, by reduction of or complete removal of typically used compression aid microcrystalline cellulose. Reduction or removal of microcrystalline cellulose results in smaller tablets, which are easier to swallow and likely to be more desirable by patients in particular those with swallowing difficulty. These tablets have faster erosion rate relative to the tablet formulation containing higher amount of microcrystalline cellulose and surprisingly no notable impact on tablet compression profile. Tablet formulation containing 10 mg of the peptide drug (Compound 1, Compound 2) and 300 mg or 600 mg of SNAC, without addition of microcrystalline cellulose, and using typical amount of Magnesium Stearate as lubricant, is shown to provide the faster dissolution described above.

Furthermore, tablets with film-coating are also developed using both slower and faster dissolution cores, aiming to provide final product with better handleability and swallowability.

Although the concept is tested using the two peptides described, it can be applied to other peptides and with variation in amount of lubricant as required for processing, typically ranging from 0.5% to 5%. If necessary, for optimum manufacturability upon scale-up, appropriate amount of microcrystalline cellulose is added to achieve a robust tablet formulation that results in concurrent release that is within the range described by the slower and faster tablets mentioned above. Addition of mannitol or other diluent instead of or in addition to microcrystalline cellulose that can enable a robust manufacturing process while maintaining the desired dissolution profile is also an option.

Examples of the aforementioned dosage forms, their method of preparation, and dissolution data are provided below.

### Composition of Examples 1-6: Tablets Containing Compound 1 and Different Amounts of SNAC

Tablets containing Compound 1 and different amounts of SNAC are prepared by blending the ingredients in a suitable blender and then compressing them into tablets using appropriate tableting equipment. At small-scale, all the components are weighed and transferred into a mortar. After mixing using a pestle for 5-10 min, the mixture from the mortar is transferred into a vial and blended further for 5-10 min in Turbula mixer. Target weight of this blend is added into a die that is installed along with appropriate punches on a manual single station hydraulic press and compressed into tablets. The composition, tablet weight, tablet tooling size and target compression pressures for each of the Compound 1 tablet examples are shown in Table 1. The blending conditions, tablet tooling and tableting parameters are further modified upon scale-up for achieving optimum manufacturability and required tablet attributes depending upon the scale and manufacturing equipment utilized.

TABLE 1
Composition and Tableting Parameters for Tablet Formulation
Examples 1-6 Containing Compound 1

| | Amount per Tablet (mg) | | | | | |
|---|---|---|---|---|---|---|
| | Ex- | Ex- | Ex- | Ex- | Ex- | Ex- |
| | ample | ample | ample | ample | ample | ample |
| Component | 1 | 2 | 3 | 4 | 5 | 6 |
| Compound 1 | 10.00ª | 10.00^{b} | 10.00ª | 10.00^{b} | 10.00ª | 10.00^{b} |
| SNAC | 300.00 | 300.00 | 600.00 | 600.00 | 300.00 | 300.00 |
| MCC | 86.00ª | None | 169.00ª | None | 86.00ª | None |
| Magnesium | 4.00 | 3.10 | 8.00 | 6.17 | 4.00 | 3.10 |
| Stearate | | | | | | |
| Total Tablet | 400.00 | 313.10^{b} | 787.00 | 616.17^{b} | 400.00 | 313.10^{b} |
| Weight Target | | | | | | |
| (mg) | | | | | | |
| Tablet Tooling | 10 mm | 10 mm | 12 mm | 12 mm | 10 mm | 10 mm |
| Size | Round | Round | Round | Round | Round | Round |
| Target | 191 | 191 | 159 | 159 | 96 | 96 |
| Compression | | | | | | |
| Pressure (MPa) | | | | | | |
| Average Solid | 0.92 | 0.90 | 0.92 | 0.90 | 0.86 | 0.82 |
| Fraction | | | | | | |

^{a}Amount of drug is corrected for potency and correspondingly amount of microcrystalline cellulose is adjusted to maintain constant total tablet weight
^{b}Amount of drug is corrected for potency and total tablet weight is adjusted accordingly

Dissolution testing of tablets described in Examples 1-6 is performed using USP Apparatus 2 (equipped with 1 L vessel and matching paddle) containing 500 mL of 50 mM pH 6.8 phosphate buffer at 37° C. and a paddle speed of 75 rpm. The amount of Compound and SNAC released are measured by HPLC. Greater than 80% concurrent release of the drug and SNAC is achieved within 30 minutes for Examples 2, 4 and 6 and within 45 minutes for Examples 1, 3 and 5. Tables 2A, B show the results of these dissolution testing for tablets described in Examples 1-6.

TABLE 2A, B
Results of Dissolution Testing for Tablets Described in
Examples 1-6 Containing Compound 1
Table 2A

| | Example 1 | | Example 2 | | Example 3 | |
|---|---|---|---|---|---|---|
| | % Release | | % Release | | % Release | |
| Time | Compound | | Compound | | Compound | |
| (min) | 1 | SNAC | 1 | SNAC | 1 | SNAC |
| 0 | 0.0 | 0.0 | 0.0 | 0.0 | 0.0 | 0.0 |
| 3 | 15.3 | 22.3 | 34.5 | 46.9 | 10.2 | 15.0 |
| 6 | 31.4 | 40.6 | 59.3 | 73.0 | 22.8 | 27.6 |
| 9 | 46.0 | 56.1 | 83.2 | 96.5 | 32.7 | 39.0 |
| 15 | 69.2 | 79.4 | 98.9 | 109.1 | 49.8 | 53.2 |
| 21 | 82.3 | 92.5 | 102.2 | 111.5 | 64.2 | 64.8 |
| 30 | 92.5 | 103.0 | 102.4 | 112.2 | 76.1 | 76.2 |
| 45 | 99.1 | 108.9 | 99.8 | 111.9 | 90.3 | 86.7 |

TABLE 2B

| | Example 4 | | Example 5 | | Example 6 | |
|---|---|---|---|---|---|---|
| | % Release | | % Release | | % Release | |
| Time | Compound | | Compound | | Compound | |
| (min) | 1 | SNAC | 1 | SNAC | 1 | SNAC |
| 0 | 0.0 | 0.0 | 0.0 | 0.0 | 0.0 | 0.0 |
| 3 | 11.9 | 19.3 | 11.1 | 20.5 | 29.2 | 41.5 |
| 6 | 25.7 | 36.6 | 25.9 | 34.9 | 57.1 | 72.9 |
| 9 | 39.2 | 51.0 | 38.1 | 47.2 | 74.8 | 84.5 |
| 15 | 61.3 | 73.5 | 56.0 | 65.1 | 88.5 | 98.6 |
| 21 | 72.3 | 88.4 | 71.7 | 81.0 | 93.4 | 104.2 |
| 30 | 92.5 | 98.8 | 85.8 | 95.8 | 90.3 | 103.7 |
| 45 | 96.7 | 103.8 | 100.2 | 111.1 | 94.7 | 103.5 |

### Composition Examples 7-13: Tablets Containing Compound 2 and SNAC with Different Drug Load and Compression Pressure

Tablets containing Compound 2 and SNAC with different drug load and compression pressure are prepared using the same procedure as described for Examples 1-6, except that the blending step in Turbula mixer was not performed. The composition, tablet weight, tablet tooling size and compression pressures for each of the Compound 2 tablet example are shown in Table 3.

TABLE 3
Composition and Tableting Parameters for Tablet Formulation Examples
7-13 Containing Compound 2

| | Amount per Tablet (mg) | | | | | | |
|---|---|---|---|---|---|---|---|
| | Example | Example | Example | Example | Example | Example | Example |
| Component | 7 | 8 | 9 | 10 | 11 | 12 | 13 |
| Compound 2 | | 4.00 | 4.00 | 24.00 | 24.00 | 36.00 | 36.00 |
| SNAC | 300.00 | 300.00 | 300.00 | 300.00 | 300.00 | 300.00 | 300.00 |
| MCC | 86.00 | 92.00 | 92.00 | 97.00 | 97.00 | 110.00 | 110.00 |
| Magnesium | 4.00 | 4.00 | 4.00 | 4.00 | 4.00 | 4.00 | 4.00 |
| Stearate | | | | | | | |
| Total Tablet | 390.00 | 400.00 | 400.00 | 425.00 | 425.00 | 450.00 | 450.00 |
| Weight | | | | | | | |
| Target (mg) | | | | | | | |
| Tooling Size | 10 mm | 10 mm | 10 mm | 10 mm | 10 mm | 10 mm | 10 mm |
| | Round | Round | Round | Round | Round | Round | Round |
| Target | 191 | 191 | 280 | 191 | 280 | 191 | 280 |
| Compression | | | | | | | |
| Pressure | | | | | | | |
| (MPa) | | | | | | | |
| Average | 0.92 | 0.90 | 0.92 | 0.90 | 0.91 | 0.90 | 0.92 |
| Solid | | | | | | | |
| Fraction | | | | | | | |

Dissolution testing of a single tablet in Example 7 is conducted using USP Apparatus 2 (equipped with 100 mL vessel and matching paddle) containing 50 mL dissolution media of 0.01 N HCl (pH 2.0) that is pre-equilibrated at 37° C., and paddle speed of 75 rpm. Tablet is taken out during the dissolution test at certain time points to measure the size of the remaining part. The amount of Compound and SNAC released are measured by HPLC. The tablet erodes rather than disintegrates. At time 0, the tablet size is 10 mm. At 21 min, the tablet size is 6 mm and at 41 min, the tablet size is 3 mm. Even at low pH where the major component of the tablet (SNAC) has low solubility, complete tablet erosion is still achieved over 45 minutes.

Dissolution testing of tablets in Examples 8-13 are done using USP Apparatus 2 (equipped with 1 L vessel and matching paddle) containing 500 mL dissolution media of 50 mM pH 6.8 phosphate buffer that is pre-equilibrated at 37° C., and paddle speed of 75 rpm. As shown in Tables 4A, B, Compound 2 and SNAC release slowly and concurrently, and greater than 80% release is achieved within 45 minutes.

TABLE 4A
Results of Dissolution Testing for Tablets Described
in Examples 8-13 Containing Compound 2

| | Example 7 | | Example 8 | | Example 10 | |
|---|---|---|---|---|---|---|
| | % Release | | % Release | | % Release | |
| Time | Compound | | Compound | | Compound | |
| (min) | 2 | SNAC | 2 | SNAC | 2 | SNAC |
| 0 | 0.00 | 0.00 | 0.00 | 0.00 | 0.00 | 0.00 |
| 3 | 10.19 | 14.31 | 11.94 | 17.63 | 11.51 | 16.53 |
| 6 | 18.56 | 24.32 | 21.69 | 30.06 | 23.14 | 29.32 |
| 9 | 24.31 | 32.35 | 31.88 | 41.78 | 35.35 | 43.01 |
| 15 | 42.44 | 46.42 | 48.56 | 59.99 | 53.42 | 60.73 |
| 21 | 55.63 | 59.92 | N/A | N/A | 71.25 | 79.53 |
| 22 | N/A | N/A | 63.25 | 76.63 | N/A | N/A |
| 30 | 66.75 | 71.10 | 76.50 | 90.27 | 84.47 | 93.33 |
| 45 | 84.31 | 88.75 | 90.06 | 104.67 | 99.98 | 108.18 |
| 60 | 98.00 | 98.04 | 95.88 | 107.66 | 105.65 | 114.41 |

TABLE 4B

| | Example 11 | | Example 12 | | Example 13 | |
|---|---|---|---|---|---|---|
| | % Release | | % Release | | % Release | |
| Time | Compound | | Compound | | Compound | |
| (min) | 2 | SNAC | 2 | SNAC | 2 | SNAC |
| 0 | 0.00 | 0.00 | 0.00 | 0.00 | 0.00 | 0.00 |
| 3 | 10.65 | 15.82 | 11.89 | 15.50 | 11.91 | 15.63 |
| 6 | 19.99 | 25.86 | 22.31 | 27.43 | 20.28 | 25.47 |
| 9 | 28.66 | 35.17 | 33.52 | 39.30 | 29.27 | 34.95 |
| 15 | 42.27 | 49.88 | 50.45 | 56.98 | 43.21 | 50.07 |
| 21 | 56.89 | 64.83 | 63.45 | 71.40 | 54.92 | 61.92 |
| 22 | N/A | N/A | N/A | N/A | N/A | N/A |
| 30 | 71.16 | 78.49 | 78.42 | 87.64 | 70.59 | 80.21 |
| 45 | 88.67 | 98.28 | 94.18 | 102.94 | 85.16 | 94.33 |
| 60 | 98.63 | 108.56 | 102.22 | 110.41 | 94.08 | 103.75 |

### Composition Examples 14-19: Solid Dosage Forms Containing Compound 2 and Different Types and Amounts of Permeation Enhancers

The tablet Examples 14, 15, 17-19 are prepared using the same procedure as described for Examples 1-6, except that the blending step in Turbula mixer is not performed. Examples 14, 18 contain SNAC, Example 15 contains CNAC, and Examples 16, 17 and 19 contain C10. The composition, tablet weight, tablet tooling size and compression pressures for each of the Compound 2 tablet example are shown in Table 5.

The immediate release capsules (Example 16) are prepared by blending the ingredients in a suitable blender and then filling into capsule shells. At small-scale, all the components are weighed and transferred into a mortar. After mixing using a pestle for 5-10 min, target weight of this mixture is manually filled into capsule shells with multiple tamping and presses. The composition, formulation weight, and capsule size are shown in Table 5. The blending conditions, capsule size and encapsulation parameters are further modified upon scale-up for achieving optimum manufacturability depending upon the scale and manufacturing equipment utilized.

TABLE 5
Composition and Dosage Unit Details for Compound 2 Formulation
Examples 14-19

| | Amount per Tablet (mg) | | | | | |
|---|---|---|---|---|---|---|
| | Example | Example | Example | Example | Example | Example |
| Component | 14 | 15 | 16 | 17 | 18 | 19 |
| Compound 2 | 10.00 | 10.00 | 10.00 | 10.00 | 10.00 | 10.00 |
| SNAC | 300.00 | None | None | None | 600.00 | None |
| C10 | None | None | 500.00 | 500.00 | None | 300.00 |
| 5-CNAC | None | 350.00 | None | None | None | None |
| MCC | 86.00 | 86.00 | None | 143.00 | 169.00 | 86.00 |
| Magnesium | 4.00 | 4.00 | None | 7.00 | 8.00 | 4.00 |
| Stearate | | | | | | |
| Total | 400.00 | 450.00 | 510.00 | 660.00 | 787.00 | 400.00 |
| Formulation | | | | | | |
| Weight Target | | | | | | |
| (mg) | | | | | | |
| Dosage form | 10 mm | 10 mm | Size 00EL | 12 mm | 12 mm | 10 mm |
| Size | Round | Round | HPMC | Round | Round | Round |
| | | | capsule | | | |
| Target | 191 | 191 | N/A | 88 | 159 | 64 |
| Compression | | | | | | |
| Pressure (MPa) | | | | | | |
| Average Solid | 0.89 | N/A | N/A | 0.96 | 0.92 | 0.92 |
| Fraction | | | | | | |

N/A: not applicable

Dissolution testing of tablets in Example 14 and 17-19 is done using USP Apparatus 2 (equipped with 1 L vessel and matching paddle) containing 500 mL dissolution media of 50 mM pH 6.8 phosphate buffer that is pre-equilibrated at 37° C., and paddle speed of 75 rpm. The amount of Compound and PE released are measured by HPLC. As shown in Tables 6A and 6B, Compound 2 and permeation enhancers release slowly and concurrently, and greater than 80% release is achieved within 45 minutes.

Dissolution testing of the unit dosage forms in Examples 15, 16 is done using USP Apparatus 2 (equipped with 100 mL vessel and matching paddle) containing 50 mL dissolution media of 50 mM pH 6.8 phosphate buffer that is pre-equilibrated at 37° C., and paddle speed of 75 rpm. As shown in Table 6A, peptide and 5-CNAC release slowly and concurrently, and greater than 80% release is achieved within 45 minutes. For immediate release capsule (Example 16), Compound 2 and C10 release immediately and concurrently, and greater than 80% release is achieved within 30 minutes after capsule shell hydration (release starts).

TABLE 6A
Results of Dissolution Testing for Dosage Forms Described in
Examples 14-19

| | Example 14 | | Example 15 | | Example 16 | |
|---|---|---|---|---|---|---|
| | % Release | | % Release | | % Release | |
| Time | Compound | | Compound | 5- | Compound | |
| (min) | 2 | SNAC | 2 | CNAC | 2 | C10 |
| 0 | 0.00 | 0.00 | 0.00 | 0.00 | 0.00 | 0.00 |
| 3 | 14.00 | 15.80 | 15.60 | 17.07 | 0.00 | 0.00 |
| 6 | 27.50 | 30.72 | 25.53 | 27.27 | 0.00 | 0.00 |
| 9 | N/A | N/A | 34.37 | 36.02 | N/A | N/A |
| 10 | 44.00 | 47.52 | N/A | N/A | 0.00 | 0.00 |
| 15 | 58.50 | 60.85 | 53.63 | 53.80 | 3.50 | 3.11 |
| 20 | N/A | N/A | N/A | N/A | 32.70 | 30.10 |
| 21 | 72.00 | 72.17 | 68.22 | 68.54 | N/A | N/A |
| 25 | N/A | N/A | N/A | N/A | 70.20 | 63.96 |
| 30 | 85.00 | 83.98 | 77.09 | 73.24 | 113.75 | 100.29 |
| 45 | 96.00 | 93.18 | 93.85 | 87.12 | 114.25 | 101.95 |
| 60 | 104.50 | 99.27 | 101.22 | 91.91 | 113.65 | 102.22 |

TABLE 6B

| | Example 17 | | Example 18 | | Example 19 | |
|---|---|---|---|---|---|---|
| | % Release | | % Release | | % Release | |
| Time | Compound | | Compound | | Compound | |
| (min) | 2 | C10 | 2 | SNAC | 2 | C10 |
| 0 | 0.00 | 0.00 | 0.00 | 0.00 | 0.00 | 0.00 |
| 3 | 9.50 | 10.24 | 10.50 | 14.73 | 13.00 | 14.28 |
| 6 | 19.50 | 19.59 | 29.50 | 32.73 | 26.50 | 25.53 |
| 9 | N/A | N/A | 42.50 | 45.17 | 37.50 | 37.08 |
| 10 | 33.00 | 32.67 | N/A | N/A | N/A | N/A |
| 15 | 46.50 | 43.63 | 62.00 | 60.83 | 55.00 | 53.70 |
| 20 | N/A | N/A | N/A | N/A | N/A | N/A |
| 21 | 59.00 | 53.87 | 73.00 | 67.17 | 68.00 | 67.27 |
| 25 | N/A | N/A | N/A | N/A | N/A | N/A |
| 30 | 71.50 | 64.34 | 86.50 | 76.57 | 85.00 | 82.33 |
| 45 | 81.00 | 72.97 | 95.50 | 84.90 | 103.50 | 92.48 |
| 60 | 91.00 | 82.73 | 102.00 | 89.00 | 112.00 | 100.40 |

### Composition of Examples 20-24: Tablets Containing Compound 2 and SNAC with and without Film-Coating

The tablets cores are prepared using the same procedure as described for Examples 1-6, except that the blending step in Turbula mixer was not performed. The composition, tablet weight, tablet tooling size and compression pressures for each of the Compound 2 tablet example is shown in Table 7.

For film coating, a bench top pan coater is used to apply HPMC-based aqueous coating solution (Opadry 03K system) onto the tablet cores. Approximately 4%+/−1% (w/w) coating is applied to tablet cores to achieve visually pleasant coating result.

TABLE 7
Composition and Tableting Parameters for Tablet
Formulation Examples 20-24 Containing Compound 2

| | Amount per Tablet (mg) | | | | |
|---|---|---|---|---|---|
| Component | Example 20 | Example 21 | Example 22 | Example 23 | Example 24 |
| Compound 2 | 10.00^{a} | 10.00^{b} | 10.00^{a} | 10.00^{b} | 10.00^{b} |
| SNAC | 300.00 | 300.00 | 300.00 | 600.00 | 300.00 |
| MCC | 84.00^{a} | None | 84.00^{a} | None | None |
| Magnesium | 6.00 | 5.00 | 6.00 | 9.82 | 5.00 |
| Stearate | | | | | |
| Total Tablet | 400.00 | 315.00^{b} | 400.00 | 619.82^{b} | 315.00^{b} |
| Weight Target | | | | | |
| (mg) | | | | | |
| Tablet Tooling | 10 mm | 10 mm | 10 mm | 12 mm | 10 mm |
| Size | Round | Round | Round | Round | Round |
| Target | 191 | 191 | 191 | 159 | 191 |
| Compression | | | | | |
| Pressure | | | | | |
| (MPa) | | | | | |
| Average Solid | 0.93 | 0.92 | 0.91 | 0.92 | 0.91 |
| Fraction | | | | | |
| Film coating | Yes | No | No | No | Yes |

^{a}Amount of drug is corrected for potency and correspondingly amount of microcrystalline cellulose is adjusted to maintain constant total tablet weight
^{b}Amount of drug is corrected for potency and total tablet weight is adjusted accordingly

Dissolution testing of tablets of Examples 20-24 is performed using USP Apparatus 2 (equipped with 1 L vessel and matching paddle) containing 500 mL of 50 mM pH 6.8 phosphate buffer at 37° C., and paddle speed of 75 rpm. The amount of Compound and SNAC released are measured by HPLC. As shown in Tables 8A, 8B, greater than 80% concurrent release of the drug and SNAC is achieved within 30 minutes for examples 21, 23, and 24, and within 45 minutes for Examples 20 and 22. The addition of film-coating to the tablet core is not affecting the in vitro release profile.

TABLE 8A
Results of Dissolution Testing for Tablets Described
in Examples 20-24

| | Example 20 | | Example 21 | | Example 22 | |
|---|---|---|---|---|---|---|
| | % Release | | % Release | | % Release | |
| Time | Compound | | Compound | | Compound | |
| (min) | 2 | SNAC | 2 | SNAC | 2 | SNAC |
| 0 | 0.00 | 0.00 | 0.00 | 0.00 | 0.00 | 0.00 |
| 3 | 2.66 | 3.18 | 14.19 | 18.57 | 15.52 | 22.87 |
| 6 | 14.19 | 16.63 | 30.60 | 37.30 | 25.71 | 33.38 |
| 9 | 25.72 | 29.22 | 47.01 | 51.20 | 32.36 | 43.25 |
| 15 | 47.01 | 51.65 | 71.85 | 73.65 | 45.22 | 58.15 |
| 21 | 66.08 | 70.30 | 88.70 | 87.97 | 55.42 | 70.63 |
| 30 | 87.81 | 90.55 | 102.01 | 97.58 | 66.06 | 83.47 |
| 45 | 97.57 | 101.80 | 101.12 | 99.40 | 80.24 | 100.87 |
| 60 | 99.79 | 101.05 | 100.67 | 99.85 | 90.44 | 108.68 |

TABLE 8B

| | |
|---|---|
| Example 23 | Example 24 |
| % Release | % Release |

| Time | Compound | | Compound | |
|---|---|---|---|---|
| (min) | 2 | SNAC | 2 | SNAC |
| 0 | 0.00 | 0.00 | 0.00 | 0.00 |
| 3 | 19.95 | 29.18 | 22.61 | 29.57 |
| 6 | 40.79 | 53.87 | 48.32 | 60.32 |
| 9 | 56.30 | 71.63 | 67.83 | 82.37 |
| 15 | 79.36 | 101.23 | 90.88 | 115.80 |
| 21 | 94.87 | 114.16 | 103.30 | 125.55 |
| 30 | 101.52 | 126.88 | 104.18 | 128.75 |
| 45 | 101.52 | 118.15 | 104.18 | 126.58 |
| 60 | 100.64 | 122.63 | 104.18 | 131.80 |

### Composition Prepared Using Dry Granulation

Tablets are prepared by pre-blending intragranular ingredients in a suitable blender, followed by dry granulation either using slugging or roller compaction approach, milling these compacts into granules using appropriate sieve, adding the extragranular ingredients, blending and finally compressing them into tablets using appropriate tableting equipment. The peptide drug and permeation enhancer used in this example are Compound 2 and SNAC respectively. At small-scale, Compound 2, SNAC and magnesium stearate are weighed and transferred into a mortar. After mixing using a pestle for 3 min, the mixture from the mortar is transferred into a bottle and blended further for 10 min in Turbula mixer. Small amount of this blend is added into appropriate die that is installed along with matching punches on a manual single station hydraulic press and compressed using pressure of approximately 38 MPa to form thin compacts, referred to as slugs, having solid fraction of 0.67-0.74. These compacts are converted into granules by passing through 30 mesh screen by gentle hand sieving. These granules are transferred back into the bottle, and required amount of microcrystalline cellulose is added, followed by blending for 5 minutes in Turbula mixer. Finally, required amount of magnesium stearate is added and blended further for 5 min in Turbula mixer. Target weight of this blend is added into a die that is installed along with appropriate punches on a manual single station hydraulic press and compressed into tablets. The composition, tablet weight, tablet tooling size and target compression pressures for this dry granulated tablet example is shown in Table 9.

TABLE 9
Composition and Tableting Parameters for the Composition
Containing Prepared Using Dry Granulation

| | |
|---|---|
| Component | Amount per Tablet (mg) |

Intragranular Components

| | |
|---|---|
| Compound 2 | 10.00^{a} |
| Salcaprozate sodium (SNAC) | 300.00 |
| Magnesium Stearate | 3.00 |

Extragranular Components

| | |
|---|---|
| Microcrystalline cellulose | 86.00^{a} |
| Magnesium Stearate | 1.00 |
| Total Tablet Weight Target (mg) | 400.00 |
| Tablet Tooling Size | 10 mm Round |
| Target Compression Pressure (MPa) | 191 |
| Average Solid Fraction | 0.92 |

^{a}Amount of drug is corrected for potency and correspondingly amount of microcrystalline cellulose is adjusted to maintain constant total tablet weight.

Dissolution testing of these tablets is performed using USP Apparatus 2 (equipped with 1 L vessel and matching paddle) containing 500 mL of 50 mM pH 6.8 phosphate buffer at 37° C., and paddle speed of 75 rpm. Results are provided in Table 9 below:

TABLE 9
Results of Dissolution Testing for Tablets
Prepared Using Dry Granulation

| | |
|---|---|
| Time | % Release |

| (min) | Compound 2 | SNAC |
|---|---|---|
| 0 | 0.0 | 0.0 |
| 3 | 6.7 | 11.6 |
| 6 | 12.6 | 18.8 |
| 9 | 18.4 | 24.6 |
| 15 | 28.8 | 35.3 |
| 21 | 38.6 | 44.8 |
| 30 | 51.7 | 57.6 |
| 45 | 69.2 | 72.5 |
| 60 | 86.0 | 86.3 |
| 75 | 94.9 | 95.0 |
| 90 | 105.1 | 102.8 |

### Oral Formulation Studies for Compound 1 in Cynomolgus Monkeys

### Bioanalytical Method:

Plasma concentrations of Compound 1 are determined by a LC/MS method that measures intact Compound 1, peptide plus linked time extension. For each assay, Compound 1 and an IS, are extracted from 100% monkey plasma. This procedure involves initially thoroughly mixing the plasma sample (50 μl) with acetonitrile:water:formic acid (50:50:0.1; 25 μl) plus 2-propanol:methanol (50:50; 250 μl). Two distinct layers are formed upon centrifugation with Compound 1 and the IS contained in the supernatant layer. After moving 200-μL of the supernatant layer to the Sirocco Protein Precipitation plate, the plate is centrifuged (conditions). Next 600-μL of Water:Formic (100:2; 600 μl) are added to each well of the Sirocco plate, and the plate is vortexed, sealed, and centrifuged. After SPE plate conditioning, the sample from the Sirocco plate (700 μl) is transferred to a Waters, SPE plate, and centrifuged. The SPE plate, after washing, is eluted with acetonitrile:formic acid (100:2; 80 μl) into Invitrosol:water:water:formic acid 100:2 (35:15:50; 100 μl). The final sample (25 μl) is loaded onto a Waters, ACQUITY UPLC BEH C18 Column, 130 Å, (2.1 mm×100 mm, 1.7 μm). The column effluent is injected into a Sciex API 5500 mass spectrometer for detection and quantitation.

### Pharmacokinetic Studies:

Male cynomolgus monkeys are administered a single intravenous dose (0.05 mg/kg) or oral formulation (10 mg/tablet) of Compound 1. The intravenous dose is in 40 mM Tris pH 8 at a dose volume of 0.5 mL/kg. Blood is collected from each animal at pre-dose, 0.5, 3, 6, 12, 24, 72, 96, 168, 240, 336, and 504 hours post-dose for pharmacokinetic characterization.

Male and female cynomolgus monkeys are administered a single oral formulation (10 mg/tablet) of Compound 1. Blood is collected from each animal at pre-dose, 0.5, 3, 6, 12, 24, 72, 96, 168, 240, 336, and 504 hours post-dose for pharmacokinetic characterization.

### Pharmacokinetic Parameters:

TABLE 10
Individual and Mean Pharmacokinetic Parameters
Following a Single 10.2 nmol/kg (0.05 mg/kg) Intravenous
Dose to Male Cynomolgus Monkeys (n = 2)

| Compound | Animal | T_{1/2} | C₀ | AUC_{0-inf} | CL |
|---|---|---|---|---|---|
| (Dose) | ID | (hr) | (nmole/L) | (hr*nmole/L) | (mL/hr/kg) |
| Compound 2 | 1 | 94 | 248 | 19493 | 0.52 |
| (10.2 nmol/kg) | 2 | 98 | 205 | 18219 | 0.56 |
| | Mean | 96 | 226 | 18856 | 0.54 |

Abbreviations: AUC_{0-inf} = area under the curve from time 0 hours to infinity, CL = clearance, C₀ = estimated plasma concentration at time zero, T_{1/2} = half-life.

TABLE 11
Mean Pharmacokinetic Parameters Following a Single Oral Dose of
Compound 1 + 300 mg SNAC to Cynomolgus Monkeys

| Example | | T_{1/2} | Tmax | Cmax | AUC_{0-inf} | CL/F | F |
|---|---|---|---|---|---|---|---|
| Formulation | | (hr) | (hr) | (nmole/L) | (hr*nmole/L) | (mL/hr/kg) | (%) |
| Example 1 | Mean | 84 | 3.8 | 151 | 13203 | 78 | 1.00 |
| (10 mg | (n = 4) | | | | | | |
| Compound 1, | SD | 15 | 1.5 | 84.7 | 8056 | 57 | 0.60 |
| 300 mg | | | | | | | |
| SNAC/tablet) | | | | | | | |
| Example 2 | Mean | 94 | 3.1 | 253 | 23202 | 78 | 1.79 |
| (10 mg | (n = 4) | | | | | | |
| Compound 1, | SD | 11 | 2.3 | 285 | 26720 | 64 | 2.13 |
| 300 mg | | | | | | | |
| SNAC/tablet) | | | | | | | |
| Example 1 | Mean | 92 | 3 | 258 | 24141 | 45 | 2.16 |
| (10 mg | (n = 6) | | | | | | |
| Compound 1, | SD | 9.2 | 0 | 179 | 18836 | 39 | 1.77 |
| 300 mg | | | | | | | |
| SNAC/tablet) | | | | | | | |
| Example 2 | Mean | 85 | 2.6 | 132 | 9941 | 160 | 0.80 |
| (10 mg | (n = 6) | | | | | | |
| Compound 1, | SD | 10 | 1.0 | 117 | 9818 | 188 | 0.69 |
| 300 mg | | | | | | | |
| SNAC/tablet) | | | | | | | |

Abbreviations:
AUC_{0-inf} = area under the curve from time 0 hours to infinity,
CL/F = clearance/bioavailability,
Tmax = time to maximal concentration,
Cmax = maximum observed plasma concentration,
T_{1/2} = half-life,
F = oral bioavailability.

TABLE 12
Mean Pharmacokinetic Parameters Following a Single Oral Dose
Compound 1 + 600 mg SNAC to Cynomolgus Monkeys

| Compound | | T_{1/2} | Tmax | Cmax | AUC_{0-inf} | CL/F | F |
|---|---|---|---|---|---|---|---|
| (Dose) | | (hr) | (hr) | (nmole/L) | (hr*nmole/L) | (mL/hr/kg) | (%) |
| Example 3 | Mean | 83 | 5.3 | 115 | 9886 | 323 | 0.80 |
| (10 mg | (n = 4) | | | | | | |
| Compound 1, | SD | 9.7 | 4.5 | 127 | 10495 | 467 | 0.85 |
| 600 mg/tablet | | | | | | | |
| SNAC) | | | | | | | |
| Example 4 | Mean | 90 | 3 | 294 | 25268 | 29 | 2.24 |
| (10 mg | (n = 6) | | | | | | |
| Compound 1, | SD | 7.3 | 0 | 213 | 17247 | 11 | 1.27 |
| 600 mg/tablet | | | | | | | |
| SNAC) | | | | | | | |

Abbreviations:
AUC_{0-inf} = area under the curve from time 0 hours to infinity,
CL/F = clearance/bioavailability,
Tmax = time to maximal concentration,
Cmax = maximum observed plasma concentration,
T_{1/2} = half-life,
F = oral bioavailability.

### Oral Formulation Studies for Compound 2 in Cynomolgus Monkeys

### Pharmacokinetics of Compound 2 in Cynomolgus Monkeys: Intravenous Administration

### Bioanalytical Summary:

Plasma concentrations of Compound 2 are determined by a LC/MS method. Compound 2 and an internal standard (IS) are extracted from 100% monkey plasma (25 μL) using 50 mM ammonium bicarbonate. After centrifugation, the supernatant is transferred and 2B10 biotinylated antibody along with 5C9 biotinylated antibody (5 μL each) are added. The samples are then centrifuged, T1 Streptavidin beads are added (20 μL) for 30 minutes, and the analyte is eluted from the beads with 30% acetonitrile with 5% formic acid in water followed by mixing. A final sample (10 μL) containing 31% acetonitrile in 10% formic acid is loaded onto a Supelco Analytical Discovery BIO Wide Pore C5-3, 5 cm×0.1 mm for LC/MS analysis.

### Pharmacokinetics:

The plasma PK of Compound 2 is evaluated in male cynomolgus monkeys following a single IV dose (50 nmol/kg). Blood samples are collected over 504 hours. Plasma is harvested from blood samples by centrifugation and stored frozen until analysis. Plasma concentrations of Compound 2 are detected through 504 hours post-dose. PK parameters for one animal are extrapolated using the concentration versus time data up to 72 hours post-dose.

TABLE 13
Pharmacokinetic Parameters of Compound 2 in Cynomolgus Monkeys
Following a Single IV Dose of 50 nmol/kg

| Compound 2 | | | | | | |
|---|---|---|---|---|---|---|
| Dose | Animal | T_{1/2} | Tₘₐₓ | Cₘₐₓ | AUC_{0-inf} | CL |
| (nmol/kg) | ID | (hr) | (hr) | (nmol/L) | (hr*nmol/L) | (mL/hr/kg) |
| 50 | 1* | 80 | 0.5 | 1182 | NR | NR |
| | 2 | 87 | 0.08 | 926 | 84232 | 0.59 |
| | Mean | 84 | 0.3 | 1054 | 84232 | 0.59 |

Abbreviations:
AUC_{0-inf} = area under the curve from 0 to infinity;
CL = clearance;
Cₘₐₓ = maximal concentration;
Tₘₐₓ = time at maximal concentration;
T_{1/2} = elimination half-life;
NR = Not reported.
* samples for Animal 1-collected through 72 hours.

### Pharmacokinetics of Compound 2 in Cynomolgus Monkeys: Oral Administration Bioanalytical Summary:

High resolution liquid chromatography/mass spectrometry (HR-LC/MS) is used to measure the concentrations of Compound 2 in cynomolgus monkey plasma. Standards and controls are prepared in cynomolgus monkey plasma using Compound 2, and any dilutions required to bring samples into the quantitative range are also performed in control cynomolgus monkey plasma. To control assay variability, an IS is added to all the standards and samples.

For Studies with Examples 14, 15, 16, 17, 18, 19: Compound 2 and the IS are extracted from monkey plasma (50 μL) by protein precipitation using isopropyl alcohol and methanol (50:50 v/v). The samples are then centrifuged (4000 rpm for 10 minutes) and the supernatant is transferred to a Siricco Protein Precipitation Plate. After centrifugation (4000 rpm for 20 minutes), the samples are loaded on a Sep-Pak tC18 SPE microelution plate that is conditioned with 2% formic acid in water. The compounds are then washed with 2% formic acid in water and eluted using 2% formic acid in acetonitrile into a plate containing 1× Invitrosol and 1% formic acid in water prior to injecting an aliquot (20 μL) on to Advantage Armor C18, 3 μm, 30×0.5 mm for LC/MS analysis.

For Studies with Examples 20, 21, 22, 23, 24: Compound 2 and the IS are extracted from monkey plasma (50 μL) by antibody capture using biotinylated antibodies IBA395 and IBA5C9 (1:1, 2 μg/well). Samples are mixed on a plate shaker for 1 hour before adding 20 μL of high-capacity magnetic beads. Samples are then mixed for 30 minutes before washing twice with phosphate buffered saline and eluting with 100 μL of 1% formic acid in water and acetonitrile (70/30% v/v). An aliquot (20 μL) is injected on to 2× Sprite AC1842 C18, 5 μm, 40×2.1 mm for LC/MS analysis.

For studies with Composition Prepared Using Dry Granulation:

Compound 2 and the IS are extracted from monkey plasma (50 μL) by protein precipitation using methanol. The samples are then centrifuged (3000 rpm for 10 minutes) and the supernatant is transferred to a Lo-bind plate and dried at 55° C. for 1 hour or until dry. The samples are then reconstituted with 1% formic acid in water and acetonitrile (50:50 v/v) and an aliquot (20 μL) is injected on to 2× Sprite AC1842 Armor C18, 5 μm, 40×2.1 mm for LC/MS analysis.

### Pharmacokinetics:

Pharmacokinetic (PK) parameters of Compound 2 are determined after a single 10-mg oral dose of Compound 2 in different formulations to male and female cynomolgus monkeys. Blood samples are collected up to 504 hours post-dose. Plasma is harvested from blood samples by centrifugation and stored frozen until analysis. Plasma concentrations of Compound 2 are detected through 504 hours post-dose. Mean Pharmacokinetic Parameters Following a Single IV or Oral Dose of Compound 2 to Cynomolgus Monkeys are presented in Tables 14, 15, 16 and 17.

TABLE 14
Mean Pharmacokinetic Parameters Following a Single Oral Dose of
Compound 2 (10 mg or 590 nmol/kg) with SNAC
(300 or 600 mg) to Cynomolgus Monkeys

| | | Dose | | | | | CL/F | F |
|---|---|---|---|---|---|---|---|---|
| Formulation | | (nmol/ | T_{1/2} | Tₘₐₓ | Cₘₐₓ | AUC_{0-inf} | (mL/hr/ | (PO/IV) |
| Example | | kg) | (hr) | (hr) | (nmol/L) | (hr*nmol/L) | kg) | (%) |
| Example 14 | Mean | 627 | 92.1 | 5 | 402 | 24021 | 51.8 | 2.22 |
| (300 | (n = 3) | | | | | | | |
| mg/tablet | SD | 68.8 | 7.28 | 2 | 387 | 18900 | 55.0 | 1.59 |
| SNAC) | | | | | | | | |
| Example 22 | Mean | 573 | 70.4 | 5 | 690 | 39996 | 15.6 | 4.19 |
| (300 | (n = 6) | | | | | | | |
| mg/tablet | SD | 67.0 | 5.51 | 2 | 340 | 11625 | 6.10 | 1.24 |
| SNAC) | | | | | | | | |
| Example 20 | Mean | 643 | 75.1 | 6 | 476 | 43026 | 29.6 | 3.92 |
| (300 | (n = 6) | | | | | | | |
| mg/tablet | SD | 93.1 | 14.0 | 5 | 469 | 48276 | 22.5 | 4.03 |
| SNAC) | | | | | | | | |
| Example 21 | Mean | 670 | 63.3 | 2 | 499 | 35569 | 23.6 | 3.24 |
| (300 | (n = 6) | | | | | | | |
| mg/tablet | SD | 80.3 | 9.02 | 1 | 223 | 19913 | 11.6 | 2.02 |
| SNAC) | | | | | | | | |
| Example 21 | Mean | 560 | 69.7 | 4 | 786 | 54887 | 37.3 | 5.41 |
| (300 | (n = 6) | | | | | | | |
| mg/tablet | SD | 88.2 | 20.8 | 4 | 595 | 44554 | 57.4 | 4.03 |
| SNAC) | | | | | | | | |
| Example 24 | Mean | 576 | 64.2 | 3 | 678 | 47748 | 16.1 | 4.75 |
| (300 | (n = 6) | | | | | | | |
| mg/tablet | SD | 82.3 | 4.8 | 1 | 462 | 33343 | 8.31 | 2.74 |
| SNAC) | | | | | | | | |
| Example 18 | Mean | 557 | 80.1 | 9 | 596 | 38502 | 27.1 | 3.99 |
| (600 | (n = 2) | | | | | | | |
| mg/tablet | | | | | | | | |
| SNAC) | | | | | | | | |
| Example 23 | Mean | 516 | 83.0 | 5 | 996 | 77307 | 10.7 | 8.74 |
| (600 | (n = 6) | | | | | | | |
| mg/tablet | SD | 85.4 | 8.75 | 4 | 560 | 56402 | 8.65 | 5.71 |
| SNAC) | | | | | | | | |

Abbreviations:
AUC_{0-inf} = area under the curve from time 0 hours to infinity,
CL/F = apparent clearance,
Tₘₐₓ = time to maximal concentration,
Cₘₐₓ = maximum plasma concentration,
T_{1/2} = half-life,
F = bioavailability.

TABLE 15
Mean Pharmacokinetic Parameters Following a Single Oral Dose of
Compound 2 (10 mg or 569 nmol/kg) with Sodium Caprate
(C10, 300 or 500 mg) to Cynomolgus Monkeys.

| | | Dose | | | | | CL/F | F |
|---|---|---|---|---|---|---|---|---|
| Formulation | | (nmol/ | T_{1/2} | Tₘₐₓ | Cₘₐₓ | AUC_{0-inf} | (mL/hr/ | (PO/IV) |
| Example | | kg) | (hr) | (hr) | (nmol/L) | (hr*nmol/L) | kg) | (%) |
| Example 19 | Mean | 555 | 68.1 | 9 | 516 | 58503 | 118 | 5.66 |
| (300 | (n = 3) | | | | | | | |
| mg/tablet | SD | 71.8 | 16.4 | 13 | 640 | 82987 | 180 | 7.63 |
| C10) | | | | | | | | |
| Example 17 | Mean | 578 | 140 | 3 | 370 | 20131 | 83.8 | 2.16 |
| (500 | (n = 3) | | | | | | | |
| mg/tablet | SD | 51.5 | 115 | 0 | 286 | 15151 | 112 | 1.64 |
| C10) | | | | | | | | |
| Example 16 | Mean | 573 | 82.7 | 3 | 180 | 12084 | 162 | 1.31 |
| (500 | (n = 3) | | | | | | | |
| mg/capsule | SD | 55.0 | 17.3 | 0 | 227 | 14963 | 182 | 1.63 |
| C10) | | | | | | | | |

Abbreviations:
AUC_{0-inf} = area under the curve from time 0 hours to infinity,
CL/F = apparent clearance,
Tₘₐₓ = time to maximal concentration,
Cₘₐₓ = maximum plasma concentration,
T_{1/2} = half-life,
F = bioavailability.

TABLE 16
Mean Pharmacokinetic Parameters Following a Single Oral Dose of
Compound 2 (10 mg or 619 nmol/kg) with 8-(N-2-hydroxy-5-chlorobenzoyl)-
amino-caprylic acid (5-CNAC, 300 mg) to Cynomolgus Monkeys (n = 3).

| | | Dose | | | | | CL/F | F |
|---|---|---|---|---|---|---|---|---|
| Formulation | | (nmol/ | T_{1/2} | Tₘₐₓ | Cₘₐₓ | AUC_{0-inf} | (mL/hr/ | (PO/IV) |
| Example | | kg) | (hr) | (hr) | (nmol/L) | (hr*nmol/L) | kg) | (%) |
| Example 15 | Mean | 619 | 64.6 | 4 | 331 | 22112 | 31.4 | 2.07 |
| (300 | | | | | | | | |
| mg/tablet | SD | 55.9 | 4.50 | 2 | 172 | 10868 | 10.6 | 0.833 |
| CNAC) | | | | | | | | |

Abbreviations:
AUC_{0-inf} = area under the curve from time 0 hours to infinity,
CL/F = apparent clearance,
Tₘₐₓ = time to maximal concentration,
Cₘₐₓ = maximum plasma concentration,
T_{1/2} = half-life,
F = bioavailability.

TABLE 17
Mean Pharmacokinetic Parameters in Cynomolgus Monkeys Following a
Single Oral Dose of Compound 2 (10 mg or 590 nmol/kg)
with SNAC (300 mg) Prepared Using Dry Granulation

| | | Dose | | | | | CL/F | F |
|---|---|---|---|---|---|---|---|---|
| Formulation | | (nmol/ | T_{1/2} | Tₘₐₓ | Cₘₐₓ | AUC_{0-inf} | (mL/hr/ | (PO/IV) |
| Example | | kg) | (hr) | (hr) | (nmol/L) | (hr*nmol/L) | kg) | (%) |
| Dry | Mean | 529 | 72.0 | 2.5 | 207 | 16364 | 34.1 | 1.86 |
| Granulation | | | | | | | | |
| (300 | | | | | | | | |
| mg/tablet | SD | 32.9 | 10.6 | 1.0 | 25.0 | 3914 | 10.3 | 0.54 |
| SNAC) | | | | | | | | |

Although dry granulation processes are more commonly used for tablets containing materials with poor physical properties and/or low doses, the exemplary compositions prepared with Compound 2 and SNAC using direct compression resulted in higher bioavailability as compared to the composition prepared with Compound 2 and SNAC using dry granulation.

### Clinical Trial

A multiple-ascending dose study is conducted to study the safety, tolerability, and pharmacokinetics of erodible tablets containing Compound 2 administered as 3 consecutive, once-daily oral doses in healthy participants. Tablets are prepared having the composition set forth in Table 18 below using a direct compression process as described for Example 22 in Table 7 above. Average solid fraction of tablets was 0.90.

TABLE 18
Tablets prepared for use in clinical trial.

Quantity (mg/tablet)

| Ingredient | 4 mg | 12 mg |
|---|---|---|
| Compound 2 | 4.000 | 12.00 |
| SNAC | 300.0 | 300.0 |
| MCC | 90.0 | 82.00 |
| Magnesium stearate | 6.000 | 6.000 |
| Total Tablet Weight | 400.0 | 400.0 |

The Compound 2 oral doses for this study are summarized in the following 4 “dose cohorts”:
Cohort 1, 4-mg dose: 1 tablet of 4 mg Compound 2+300 mg SNAC
Cohort 2, 8-mg dose:2 tablets of 4 mg Compound 2+300 mg SNAC
Cohort 3, 12-mg dose: 1 tablet of 12 mg Compound 2+300 mg SNAC
Cohort 4, 24-mg dose:2 tablets of 12 mg Compound 2+300 mg SNAC

Sufficient participants are randomly assigned to study intervention to ensure approximately 10 evaluable participants (8 receiving Compound 2 and 2 receiving placebo) from each of 4 dose cohorts complete the study. In each cohort, eligible participants are randomly assigned to receive 3 once-daily doses of either Compound 2 or placebo. Data for the 4 cohorts are provided in Table 18 below, along with a comparison to IV data from a separate study having similar protocol design and conducted at the same clinical site.

TABLE 18
Dose-Normalized Compound 2 AUC(0-168) and AUC(0-∞)

| | | Oral 8 mg + | Oral 24 mg + | Oral 12 mg + | Oral 4 mg + |
|---|---|---|---|---|---|
| | | 600 mg | 600 mg | 300 mg | 300 mg |
| Parameter | IV 0.5 mg | SNAC | SNAC | SNAC | SNAC |
| Dose-Normalized | 22800^{e} | 587 | 683 | 617 | 460 |
| AUC(0-168) | | | | | |
| (ng · h/mL · mg)^{a} | | | | | |
| Oral | 100 | 2.58 | 2.99 | 2.71 | 2.02 |
| Bioavailability | | | | | |
| Based on AUC | | | | | |
| (0-168) (%)^{b} | | | | | |
| Dose-Normalized | 49200^{e} | 1420 | 1630 | 1570 | 1060 |
| AUC(0-∞) | | | | | |
| (ng · h/mL · mg)^{c} | | | | | |
| Oral | 100 | 2.89 | 3.32 | 3.20 | 2.15 |
| Bioavailability | | | | | |
| Based on AUC | | | | | |
| (0-∞) (%)^{d} | | | | | |

Abbreviations: AUC(0-168) = area under the concentration versus time curve from time zero to 168 h; AUC(0-∞) = area under the concentration versus time curve from time zero to infinity; IV = intravenous; SNAC = salcaprozate sodium.
^{a}Dose-normalized AUC(0-168) = AUC(0-168)/Total oral Compound 2 dose over 3 days
^{b}Oral bioavailability based on AUC(0-168) = Dose-normalized oral AUC(0-168)/Dose-normalized iv AUC(0-168)
^{c}Dose-Normalized AUC(0-∞) = AUC(0-∞)/Total oral Compound 2 dose over 3 days
^{d}Oral bioavailability based on AUC(0-∞) = Dose-normalized oral AUC(0-∞)/Dose-normalized iv AUC(0-∞)

We claim:

1. An erodible tablet for oral administration wherein the erodible tablet comprises a therapeutic peptide or a pharmaceutically acceptable salt thereof;
a permeation enhancer; and
a lubricant, wherein the average solid fraction of the tablet is between 0.75 and 0.98.

2. An erodible tablet according to claim 1, wherein the average solid fraction of the tablet is between 0.8 and 0.98.

3. An erodible tablet according to claim 1, wherein the average solid fraction of the tablet is between 0.82 and 0.96.

4. An erodible tablet according to claim 1 further comprising microcrystalline cellulose (MCC).

5. An erodible tablet according to claim 4, wherein the MCC of the tablet is up to a maximum of 175 mg.

6. An erodible tablet according to claim 4, wherein the MCC of the tablet is up to a maximum 169 mg.

7. An erodible tablet according to claim 4, wherein the MCC of the tablet is about 30 to about 90 mg.

8. An erodible tablet according to claim 1, wherein the permeation enhancer in the tablet is Sodium N-[8-(2-hydroxybenzoyl)amino]caprylate (SNAC), Salcaprozate Sodium, Sodium Caprate (C10), or 8-(N-2-hydroxy-5-chlorobenzoyl)-amino-caprylic acid (5-CNAC).

9. An erodible tablet according to claim 8, wherein the permeation enhancer is SNAC between about 300 and 600 mg.

10. An erodible tablet according to claim 9, wherein the SNAC in the tablet is 300 mg or 600 mg.

11. An erodible tablet according to claim 8, wherein the permeation enhancer is C10 between 300 and 500 mg.

12. An erodible tablet according to claim 11, wherein the C10 in the tablet is 300 mg or 500 mg.

13. An erodible tablet according to claim 8, wherein the permeation enhancer is 5-CNAC and wherein the 5-CNAC in the tablet is about 500 mg.

14. An erodible tablet according to claim 1, wherein the lubricant is magnesium stearate.

15. An erodible tablet according to claim 14, wherein the magnesium stearate in the tablet is between 3 and 30 mg.

16. An erodible tablet according to claim 1, wherein the therapeutic peptide in the tablet is between 1 and 50 mg.

17. An erodible tablet according to claim 16, wherein the therapeutic peptide in the tablet is between 1 and 36 mg.

18. An erodible tablet according to claim 16, wherein the therapeutic peptide has agonistic activity at one or more of the glucose-dependent insulinotropic polypeptide (GIP) receptor, the glucagon-like peptide-1 (GLP-1) receptor and the glucagon (GCG) receptor.

19. An erodible tablet according to claim 18, wherein the therapeutic peptide has agonistic activity at each of a glucose-dependent insulinotropic polypeptide (GIP) receptor and glucagon-like peptide-1 (GLP-1).

20. An erodible tablet according to claim 18, wherein the therapeutic peptide further has glucagon (GCG) receptor agonistic activity.

21. An erodible tablet according to claim 19, wherein the therapeutic peptide is Compound 1, or a pharmaceutically acceptable salt thereof.

22. An erodible tablet according to claim 19, wherein the therapeutic peptide is Compound 2, or a pharmaceutically acceptable salt thereof.

23. An erodible tablet according to claim 1, wherein the therapeutic peptide and the permeation enhancer are released concurrently.

24. An erodible tablet according to claim 23, wherein greater than 80% concurrent release of the therapeutic peptide and permeation enhancer is achieved.

25. An erodible tablet according to claim 1, wherein greater than 80% release of the therapeutic peptide and the permeation enhancer is achieved within 60 minutes.

26. An erodible tablet according to claim 25, wherein greater than 80% release of the therapeutic peptide and the permeation enhancer is achieved within 45 minutes.

27. An erodible tablet according to claim 1, wherein greater than 80% release of the therapeutic peptide and the permeation enhancer is achieved within 30 minutes.

28. An erodible tablet according to claim 1, wherein the tablet core is film-coated with 4%±1% (w/w) coating.

29. A cosmetic composition comprising an erodible tablet according to claim 1, wherein the tablet is film-coated with 4%±/−1% (w/w) coating.

30. A method of manufacturing an erodible tablet comprising blending a therapeutic peptide or a pharmaceutically acceptable salt thereof, a permeation enhancer, a lubricant and, optionally microcrystalline cellulose, and compressing the blended constituents to achieve an average solid fraction between 0.75 and 0.98.

31. A method according to claim 30 wherein the average solid fraction of the tablet is between 0.8 and 0.98.

32. A method according to claim 31 wherein the average solid fraction of the tablet is between 0.82 and 0.96.

33. An erodible tablet for oral administration produced by the method according to claim 30.

34. An erodible tablet for oral administration according to claim 33 wherein the erodible tablet comprises a therapeutic peptide or a pharmaceutically acceptable salt thereof;
a permeation enhancer; and
a lubricant, wherein the average solid fraction of the tablet is between 0.75 and 0.98.

35. A method of treating diabetes comprising the steps of:
orally administering to an individual in need thereof an erodible tablet according to claim 1.

36. A method of treating diabetes according to claim 35, wherein the erodible tablet is administered once daily, twice daily, alternate days, every third day, every fourth day, every fifth day, every sixth day or once weekly.

37. A method of treating diabetes according to claim 36, wherein the erodible tablet is administered once daily.

38. A method of treating obesity comprising the steps of:
orally administering to an individual in need thereof an erodible tablet according to claim 1.

39. A method of treating obesity according to claim 38, wherein the erodible tablet is administered once daily, twice daily, alternate days, every third day, every fourth day, every fifth day, every sixth day or once weekly.

40. A method of treating obesity according to claim 39, wherein the erodible tablet is administered once daily.

41. A method of treating at least one condition selected from the group consisting of diabetes mellitus, dyslipidemia, fatty liver disease, metabolic syndrome, non-alcoholic steatohepatitis, obesity and prevention of cognitive decline comprising administering an erodible table of claim 1 to a patient in need thereof.

42. An erodible tablet of claim 1 for use in the treatment of diabetes mellitus, dyslipidemia, fatty liver disease, metabolic syndrome, non-alcoholic steatohepatitis, obesity and prevention of cognitive decline.

43. An erodible tablet of claim 1 for use in the treatment of type II diabetes mellitus.

44. An erodible tablet of any one of claim 1 for use in the treatment of obesity.


SEQUENCE LISTING (5 sequences)

SEQ ID NO: 1 - protein, 39 aa, Artificial Sequence
  Synthetic Construct
  MISC_FEATURE (1)..(1): Xaa at position 1 is D-Tyr
  MISC_FEATURE (2)..(2): Xaa at position 2 is Aib
  MISC_FEATURE (13)..(13): Xaa at position 13 is alpha-methyl-Leu
  MOD_RES (17)..(17): Lys at position 17 is chemically modified by conjugation of the epsilon-amino group of the Lys side chain with (2-[2-(2-amino-ethoxy)-ethoxy]-acetyl)2-(gamma-Glu)-CO-(CH2)18-CO 2H
  MISC_FEATURE (20)..(20): Xaa at position 20 is Aib
  MISC_FEATURE (25)..(25): Xaa at position 25 is alpha-methyl-Tyr
  MOD_RES (39)..(39): Ser at position 39 is amidated
XXEGTFTSDYSIXLDKKAQXEFIEXLIAGGPSSGAPPPS

SEQ ID NO: 2 - protein, 39 aa, Artificial Sequence
  Synthetic Construct
  MISC_FEATURE (2)..(2): Xaa at position 2 is Aib
  MISC_FEATURE (6)..(6): Xaa at position 6 is alpha-MeF(2F)
  MISC_FEATURE (10)..(10): Xaa at position 10 is 4Pal
  MISC_FEATURE (13)..(13): Xaa at position 13 is alpha-MeL
  MISC_FEATURE (16)..(16): Xaa at position 16 is Orn
  MOD_RES (17)..(17): K at position 17 is chemically modified by conjugation of the epsilon-amino group of the K side chain with (2-[2-(2-amino-ethoxy)-ethoxy]-acetyl)2-(gamma-Glu)-CO-(CH2)16-CO 2H
  MISC_FEATURE (20)..(20): Xaa at position 20 is Aib
  MISC_FEATURE (24)..(24): Xaa at position 24 is D-Glu
  MISC_FEATURE (25)..(25): Xaa at position 25 is alpha-MeY
  MOD_RES (39)..(39): The Serine at position 39 is amidated
YXEGTXTSDXSIXLDXKAQXEFIXXLIEGGPSSGAPPPS

SEQ ID NO: 3 - protein, 31 aa, Homo sapiens
HAEGTFTSDVSSYLEGQAAKEFIAWLVKGRG

SEQ ID NO: 4 - protein, 42 aa, Homo sapiens
YAEGTFISDYSIAMDKIHQQDFVNWLLAQKGKKNDWKHNITQ

SEQ ID NO: 5 - protein, 29 aa, Homo sapiens
HSQGTFTSDYSKYLDSRRAQDFVQWLMNT